(19) United States
(12) Patent Application Publication
(10) Pub. No.: US 2024/0197944 A1
(43) Pub. Date: Jun. 20, 2024

(54) Sensor Device

(71) Applicant: 3M INNOVATIVE PROPERTIES COMPANY, St. Paul, MN (US)

(72) Inventors: Wensheng Xia, Woodbury, MN (US); Naiyong Jing, St. Paul, MN (US); James A. Phipps, River Falls, WI (US)

(21) Appl. No.: 18/555,642

(22) PCT Filed: Mar. 30, 2022
(86) PCT No.: PCT/IB2022/052970

§ 371 Date: Oct. 16, 2023

Related U.S. Application Data
(60) Provisional application No. 63/180,957, filed on Apr. 28, 2021.
(60) Provisional application No. 63/268,006, filed on Feb. 15, 2022.

(51) Int. Cl. A61L 2/28; A61L 2/07; A61L 2/20; G01N 27/12

(52) U.S. Cl. CPC A61L 2/28; G01N 27/126; A61L 2/07; A61L 2/208

(57) ABSTRACT

A sensor device includes a first electrode and a second electrode. Each of the first and second electrodes are electrically coupled to an electrical bridge. The electrical bridge includes a conductive polymer having a first impedance state and a second impedance state that is different than the first impedance state. The electrical bridge includes metal or metal containing particles.

### BACKGROUND

Sensor devices useful for sterilization monitoring have been described in, for example, U.S. Pat. Nos. 8,492,162, 4,594,223, and 5,204,062.

### SUMMARY

In some embodiments, the sensor device can directly report the pass/fail information regarding sterilization cycles to avoid any subject human eye color judgement to reduce errors. Also, the digitalization of chemical indicator will free people from subjective interpretation of results, manual document and physical storage.

In one aspect, the present disclosure provides a sensor device comprising: a sterilant-responsive switch comprising: a first electrode and a second electrode, each having a first end electrically coupled to the circuit and a second end; a conductive polymer having a first state and a second state; a conductive particle; and a polymeric binder, wherein the conductive polymer is capable of being converted from being in the first state to being in the second state when in contact with a sterilant.

In another aspect, the present disclosure provides a method, the method comprising: providing the sensor device of the present disclosure; exposing the sensor device to a sterilant in a sterilization process; allowing the sterilant-responsive switch to react with the sterilant which changes the sterilant-responsive switch from the first state to the second state.

In another aspect, the present disclosure provides a system, the system comprising: the sensor device of the present disclosure; a memory element to store data captured by the sensor device; and a sensing device configured to interrogate the sensor device.

Various aspects and advantages of exemplary embodiments of the present disclosure have been summarized. The above Summary is not intended to describe each illustrated embodiment or every implementation of the present disclosure. Further features and advantages are disclosed in the embodiments that follow. The Drawings and the Detailed Description that follow more particularly exemplify certain embodiments using the principles disclosed herein.

### Definitions

For the following defined terms, these definitions shall be applied for the entire Specification, including the claims, unless a different definition is provided in the claims or elsewhere in the Specification based upon a specific reference to a modification of a term used in the following definitions:

The terms “about” or “approximately” with reference to a numerical value or a shape means+/−five percent of the numerical value or property or characteristic, but also expressly includes any narrow range within the +/−five percent of the numerical value or property or characteristic as well as the exact numerical value. For example, a temperature of “about” 100° C. refers to a temperature from 95° C. to 105° C., but also expressly includes any narrower range of temperature or even a single temperature within that range, including, for example, a temperature of exactly 100° C. For example, a viscosity of “about” 1 Pa-sec refers to a viscosity from 0.95 to 1.05 Pa-sec, but also expressly includes a viscosity of exactly 1 Pa-sec. Similarly, a perimeter that is “substantially square” is intended to describe a geometric shape having four lateral edges in which each lateral edge has a length which is from 95% to 105% of the length of any other lateral edge, but which also includes a geometric shape in which each lateral edge has exactly the same length.

The term “substantially” with reference to a property or characteristic means that the property or characteristic is exhibited to a greater extent than the opposite of that property or characteristic is exhibited. For example, a substrate that is “substantially” transparent refers to a substrate that transmits more radiation (e.g. visible light) than it fails to transmit (e.g. absorbs and reflects). Thus, a substrate that transmits more than 50% of the visible light incident upon its surface is substantially transparent, but a substrate that transmits 50% or less of the visible light incident upon its surface is not substantially transparent.

The terms “a”, “an”, and “the” include plural referents unless the content clearly dictates otherwise. Thus, for example, reference to a material containing “a compound” includes a mixture of two or more compounds.

“Ionic salt” refers to any salt having a cation selected from a group I, group II metal (particularly an alkaline earth metal), or post-transition metal. Preferably, magnesium or bismuth.

The anions of an ionic salt can be selected from halogens, oxygen, sulfur, carbonate, borate, titanate, molybdate, phosphate, oxychloride, or combinations thereof.

“Integrated circuit” refers to a component that stores and processes information, in particular, a component that modulates and demodulates radio-frequency (RF) signals

“Post-transition metal” refers to post-transition metals are a set of metallic elements in the periodic table located between the transition metals to their left, and the metalloids to their right. As suggested by, Huheey J E, Keiter E A & Keiter R L 1993, Chapter 14, Principles of Structure & Reactivity, 4th ed., HarperCollins College Publishers, ISBN 0-06-042995-X, includes Ga, In, Tl, Sn, Pb, Bi, Al, Ge, Sb, Po.

“Second substrate position” refers to a position on the substrate that indicates adequate sterilization. May be established partially by the wicking substrate.

“Conductive element” refers to refers to an ability to conduct an electric current. Electrically conductive materials have an electrical conductivity of at least 2 Siemens per centimeter. Exemplary conductive elements include silver, gold, copper, aluminum, carbon black or combinations thereof.

“Monitoring loop” refers to an open or closed electrical loop.

“Adequate sterilization process” refers to a sterilization process that achieves a sterility assurance level of 10⁻⁶, or 12 log reduction of Bacillus Subtilis var. niger. The sterility assurance level is related to a probability that a sterilized unit remains nonsterile after undergoing the sterilization process.

“Wicking” refers to any suitable material through which the organic compound can migrate by capillary action. Wicking substances can include paper strips, non-woven polymeric fabrics and inorganic fibrous compositions. Preferred wicking substances are Whatman No. 1 filter paper, Whatman No. 114 filter paper, PET fabric nonwoven, supported microcrystalline cellulose (TLC plate), supported aluminum oxide, and supported silica gel.

“Adequate environmental condition” refers to environmental conditions inside of a sterilization chamber that correspond to the adequate sterilization process.

“Conductive trace” refers to a conductive element forming part of an electrical circuit. Can also be a wire.

The phrase “comprises at least one of” followed by a list refers to comprising any one of the items in the list and any combination of two or more items in the list. The phrase “at least one of” followed by a list refers to any one of the items in the list or any combination of two or more items in the list.

Although the term “impedance” is used, the term “impedance” is the reciprocal of the “admittance”. Depending on the context, either impedance or admittance can be used as changes in the impedance of a material also change the admittance of the material.

The term “and/or” means one or all of the listed elements or a combination of any two or more of the listed elements.

All numerical ranges are inclusive of their endpoints and nonintegral values between the endpoints unless otherwise stated (e.g., 1 to 5 includes 1, 1.5, 2, 2.75, 3, 3.80, 4, 5, etc.).

These and other aspects of the present disclosure will be apparent from the detailed description below. In no event, however, should the above summaries be construed as limitations on the claimed subject matter, which subject matter is defined solely by the attached claims, as may be amended during prosecution.

### BRIEF DESCRIPTION OF THE DRAWINGS

The disclosure may be more completely understood in consideration of the following detailed description of various embodiments of the disclosure in connection with the accompanying figures, in which:

FIG. 1 illustrates a sterilization system that can be used in connection with the sensors of the present disclosure.

FIG. 2 illustrates a sensor device in accordance with some embodiments of the present disclosure.

FIG. 3 illustrates use of a sensor device in a sterilization system in accordance with some embodiments of the present disclosure.

FIG. 4 illustrates use of a sensor device in a sterilization system in accordance with some embodiments of the present disclosure.

FIG. 5 illustrates a sterilization indicator system in accordance with one embodiment.

FIG. 6A illustrates a sterilization indicator sensor in accordance with one embodiment.

FIG. 6B illustrates an alternative sterilization indicator sensor in accordance with one embodiment.

FIG. 7 illustrates a sterilization indicator sensor at a different view.

FIG. 8 illustrates a method in accordance with one embodiment.

While the above-identified drawings, which may not be drawn to scale, set forth various embodiments of the present disclosure, other embodiments are also contemplated, as noted in the Detailed Description. In all cases, this disclosure describes the presently disclosed invention by way of representation of exemplary embodiments and not by express limitations. It should be understood that numerous other modifications and embodiments can be devised by those skilled in the art, which fall within the scope and spirit of this disclosure.

### DETAILED DESCRIPTION

Chemical indicators are widely used in sterilization monitoring to ensure the sterilization process has been completed correctly. Failed or insufficient sterilization cycles will put the patients in huge risk due to the potential cross-contaminations from the reprocessed surgical instruments.

Traditional chemical indicators are based on colorimetric changes in the presence of a certain sterilant and its running conditions such as sterilization temperature and sterilization time, etc. For example, a steam indicator may change color from light yellow to black. Another type chemical indicator such as Bowie-Dick test pack is designed to detect air leak or insufficient air removal in a sterilizer.

In the current practice of evaluating a chemical indicator visually, a user needs to visually judge the color development to determine if the chemical indicator was subjected to an adequate sterilization process. However, color development can be subjective. As a result, a better system is highly desirable.

Before any embodiments of the present disclosure are explained in detail, it is understood that the present disclosure is not limited in its application to the details of use, construction, and the arrangement of components set forth in the following description. The disclosure is capable of other embodiments and of being practiced or of being carried out in various ways that will become apparent to a person of ordinary skill in the art upon reading. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized and structural or logical changes may be made without departing from the scope of the present disclosure.

Unless otherwise indicated, all numbers expressing quantities or ingredients, measurement of properties and so forth used in the Specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

In some embodiments, the present disclosure relates to a sterilization system and associated sensor device having a sterilant-responsive switch that may be responsive to environmental conditions (including the presence of a sterilant such as steam) in a sterilization process. Generally, the sensor devices of the present disclosure enable electronical reporting of information (e.g., pass/fail information, accept/reject information) regarding each sterilization cycle to avoid subjective judgements that can lead to errors (e.g., perceived change in color by the human eye). Also, the systems and devices of the present disclosure enable digitalization of sterilization results which, in turn, will free technicians from manual document and physical storage.

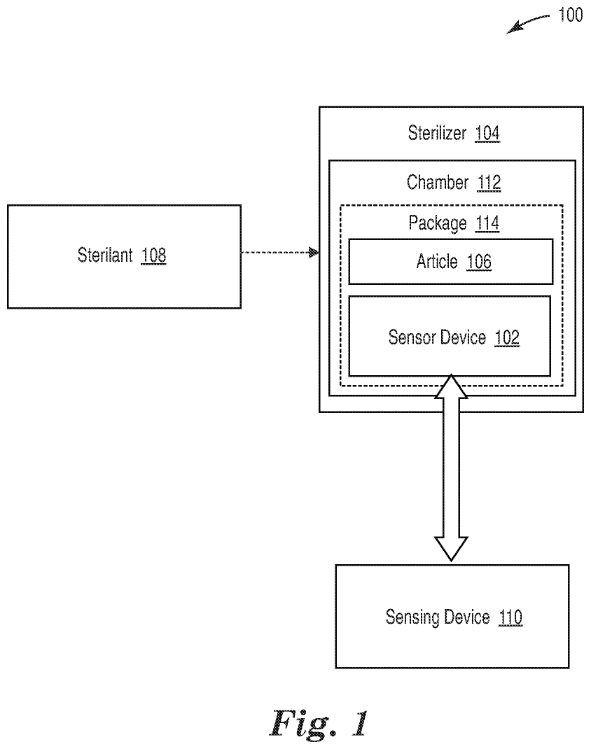

FIG. 1 illustrates a sterilization system 100 in which a sensor device of the present disclosure may be employed. As shown in FIG. 1, the sterilization system 100 may include a chamber 110 into which a sterilant stream 120 may be directed. The sterilization system 100 may be of a type commonly used by hospitals and other medical facilities to sterilize reusable medical devices. Various types of sterilization systems 100 can be employed for purposes of the present disclosure. For example, the sterilization systems 100 can be based on steam or hydrogen peroxide (e.g., vaporized hydrogen peroxide), and each type can have different sterilization process conditions. Examples of sterilizer systems using hydrogen peroxide as a sterilant are commercially available from Steris (Mentor, OH) or Tuttnauer (Israel). Examples of sterilizers using steam as a sterilant are commercially available from Steris (Mentor, OH) or Getinge (Gothenburg, Sweden).

In some embodiments, the chamber 110 can have one or more environmental conditions. The environmental conditions can be related to conditions inside of the chamber 110 and can include, for example, exposure time, sterilant (presence, concentration, etc.), temperature, pressure, or combinations thereof. In some embodiments, a first environmental condition can exist pre-sterilization process and a second environmental condition can exist during the sterilization process.

In some embodiments, the present disclosure is directed to a sensor device that is configured to determine whether a sterilization process within a sterilization system is carried out in accordance with a predetermined guideline or whether an adequate sterilization process was achieved. An adequate sterilization process can vary based on the sterilant used, the manufacturer of the sterilizer, or the articles to be sterilized. For example, Guideline for Disinfection and Sterilization in Healthcare Facilities, Center for Disease Control (2008), which is herein incorporated by reference in its entirety, provides minimum cycle times for sterilization of various article types and sterilants.

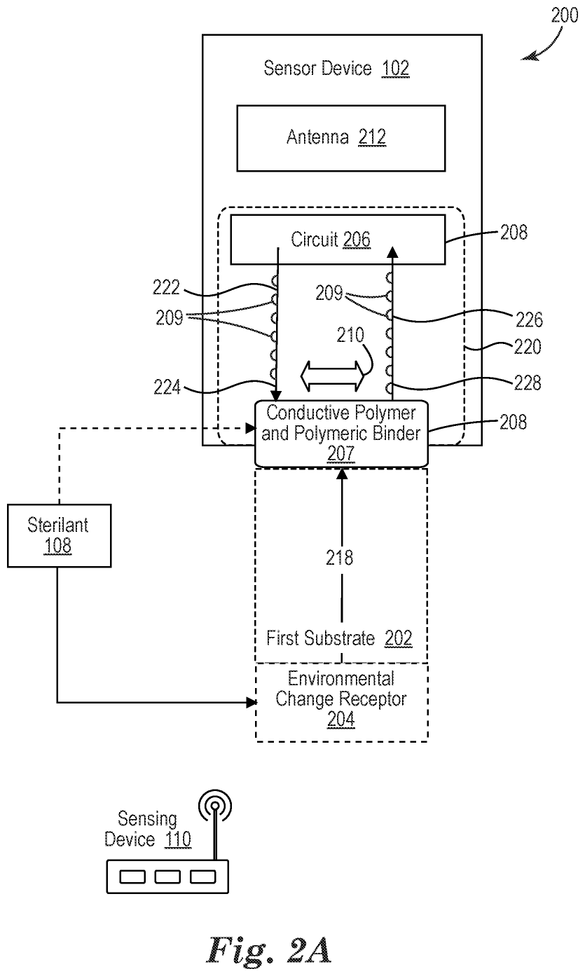

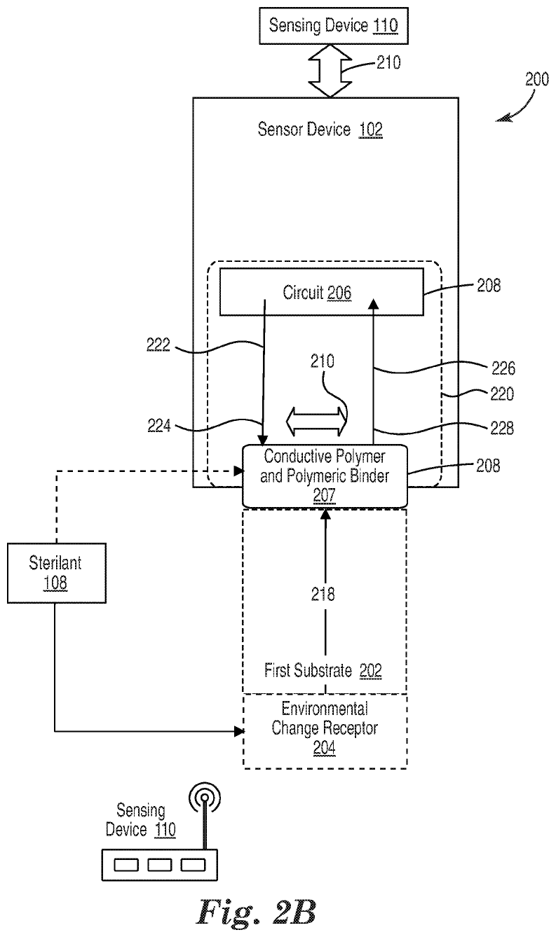

Referring to FIG. 2, a sensor device 130 in accordance with some embodiments of the present disclosure is depicted. The sensor device 130 may include a first electrode 135, a second electrode 140 (sometimes, collectively, referred to as an electrode pair), and a sterilant-responsive electrical bridge 145 which may facilitate electrical communication between the first electrode 135 and the second electrode 140. In some embodiments, each of the first electrode 135 and the second electrode 140 may be in electrical communication, or electrically coupled, (either via physical contact or via an intermediate such as a conductive member (e.g., an electrically conductive wire)) via the sterilant-responsive electrical bridge 145. As shown in FIG. 2, in some embodiments, an end of each of the first electrode 135 and the second electrode 140 may be in physical contact with the sterilant-responsive electrical bridge 145. In some embodiments, absent the sterilant-responsive electrical bridge 145, the electrode pair 135, 140 may not be capable of electrical communication (i.e., the electrodes are not physically touching or are spaced apart at least a distance such that there is no electrical communication without an intervening conductive member).

In some embodiments, the first and second electrodes 135, 140 may include a metal such as aluminum, iron, zinc, tungsten, molybdenum, tin, nickel, copper, or alloys thereof, or carbon black, graphene, carbon nanotubes, or a conducting polymer.

In some embodiments, the electrical bridge 145 may be configured to have a first impedance state (e.g., high impedance/no or low conductivity) and a second impedance state that is markedly different than the first impedance state (e.g., low impedance/high conductivity (or vice versa). For example, in some embodiments, in a first state, the electrical bridge exhibits a low impedance and in a second state exhibits a high impedance (relative to the low impedance state). In some alternative embodiments, in a first state, the electrical bridge exhibits a low electric capacitance and in a second state exhibits a high electric capacitance (relative to the low electric capacitance state) or vice versa.

In some embodiments, referring still to FIG. 2, the electrical bridge 145 may include a conductive polymer, a plurality of one or more types of metal or metal containing particles, and optionally a polymeric binder. For example, the conductive polymer and metal particles may be dispersed in a polymeric binder and deposited onto the electrode pair. As another example, the conducting polymer may be disposed in a layer that is deposited on the electrode pair and the metal particles may be present in a sterilant soluble (e.g., steam soluble) layer that is coated on the conductive polymer layer such that the metal particles will diffuse into the conducting polymer after exposure to the sterilant.

Generally, the conductive polymer material can be any polymeric material that may be shifted between a first impedance state and a second impedance state. In some embodiments, suitable conductive polymers may be those capable of being converted a first impedance state to a second impedance state in response to a change of environmental conditions (e.g., transitioning from the first state to the second state upon contact with a sterilant, or transitioning from the first state to the second state upon achievement of an adequate sterilization process within a sterilizer system). In some embodiments, the first state can be a low impedance state and the second state can be a high impedance state (or vice versa). In some embodiments, the low impedance state can be a doped (e.g., acid doped) electrically conductive state and the high impedance state can be can be a de-doped (e.g., by inclusion and activation of a basic material) electrically non-conductive (or at least a conductivity lower than that of the electrically conductive state). In some embodiments, a low impedance state refers to a state having an admittance sufficient to electrically bridge an open circuit, e.g., having an admittance of at least 2 siemens.

In some embodiments, the conductive polymer material of the electrical bridge 145 can have a repeat unit of: aniline, acetylene, pyrrole, phenylene, phenylene vinylene, phenylene ethynylene, phenylene sulfide, fluorene, pyrene, azulene, naphthalene, carbazole, indole, thiophene, ethylene dioxythiophene, or combinations thereof. The conductive polymer material can be doped or undoped with various dopants such as dinonylnaphthalene sulfonic acid (DNNSA), dodecylbenzenesulfonic acid (DBSA), arsenic pentafluoride, triiodide, camphorsulfonate, methanesulfonic acid, halogens or polyhalogen ions, methanol, hydrogen sulfate, hydrochloric acid, tetrafluoroborate, sodium sulfite, or combinations thereof.

In some embodiments, the conductive polymer material includes (or consists essentially of) polyaniline (PANI). In some embodiments, the conductive PANI is in a form of electrolytes, polyelectrolytes or PANI salts which can be readily achieved by acid-doping of PANI. PANI can be in one of three oxidation states (leucoemeraldine, emeraldine (in the salt or base forms), and per(nigraniline)). The emeraldine can be non-conductive in the base form and conductive in the polyelectrolyte form or the salt form. The emeraldine salt can be converted into the leucoemeraldine salt or per(nigraniline) which are non-conductive, via a redox reaction. The conductive polymer can be converted to non-conductive polymer via a de-doping reaction. In some embodiments, the conductive polymer material of the present disclosure may be present, initially, in the emeraldine salt form and be convertible to the leucoemeraldine salt form upon exposure to a sterilant.

In some embodiments, suitable metal or metal containing particles may include electrically conductive metal particles. Additionally, or alternatively, in some embodiments, the metal particles may be characterized as redox particles (i.e., particles that facilitate a chemical reaction in the electrical bridge 145 in the presence of a sterilant (e.g., steam) that involves loss of one or more electrons by one molecule (oxidation—metal redox particle) and simultaneous gain by another (reduction—conductive polymer)). In some embodiments, suitable metal redox particles may include aluminum, tin, bismuth, nickel, lead, Indium, chromium, gallium, iron, vanadium, cadmium, titanium, zirconium, nobelium, tungsten, thallium, germanium, or lanthanides. In some embodiments, the metal particles may include tin. In some embodiments, suitable metal particles may include metal alloy, such as silver-tin alloy, gold-tin alloy, or indium-tin alloy.

In some embodiments, useful metal redox particles may be those that can release electrons upon exposure to a sterilant (e.g., steam). For example, suitable metal redox particles may include those that can be activated to release electrons to reduce PANI electrolytes or polyelectrolytes (protonated forms) to its leucoemeraldine salt form. An examples of such a mechanism is below:

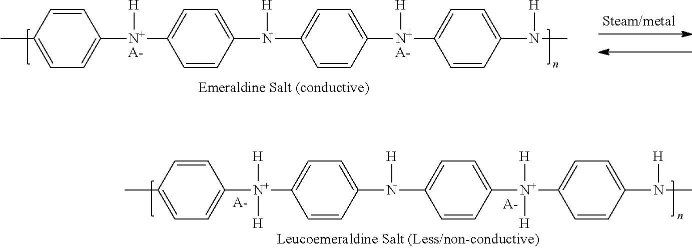

In some embodiments, suitable metal or metal containing particles may include electrically conductive metal particles, non-conductive metal oxides, metal complexes or a combination thereof, which may be characterized as catalyst particles (i.e., particles that catalyze a chemical reaction in the electrical bridge 145 in the presence of a sterilant (e.g., hydrogen peroxide) that involves the formation of byproducts that result in a local pH increase near the conductive polymer). In some embodiments, suitable metal catalyst particles may include magnesium, copper, cobalt, manganese, zinc, iron, silver, platinum, osmium, iridium, lead palladium, ruthenium, rhodium, gold, chromium, iron, vanadium, cadmium, titanium, zirconium, nobelium, tungsten, thallium or their oxidates and complexes. In some embodiments, suitable metal containing catalyst particles may include magnesium oxide, iron oxide, manganese oxide, zinc oxide, iron oxide, potassium dichromate, vanadyl acetylacetonate, 1:1 copper(II)-, manganese (II)-, cobalt(II)- or nickel(II)-hexamine complexes.

In some embodiments, useful metal catalyst particles may include those that can catalyze a reaction with the sterilant (e.g., hydrogen peroxide) to generate hydroxide anions and water as byproducts. The presence of hydroxide anions may, in turn, increase the local pH near the conductive polymer, which may result in the capture of protons to neutralize PANI electrolytes or polyelectrolytes (protonated forms) to its neutral or less protonated emeraldine form. An example of such a set of reactions (using hydrogen peroxide as the sterilant) is below:

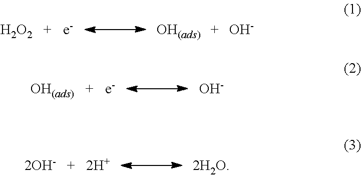

In some embodiments, useful metal or metal containing particles may include those that can be activated by a sterilant (e.g., steam or hydrogen peroxide) to generate free electrons, hydrides, or hydrogen which are capable of reducing a conductive polymer from a first conductive state to a second conductive state (e.g., converting PANI from the emeraldine salt (ES) state to the leucoemeraldine salt (LS) state). Examples of such a set of reactions are shown below (unbalanced equations):

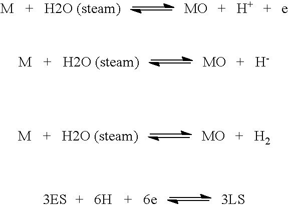

M can be monovalent or multivalent metals

In any of the above-described embodiments, the metal or metal containing particles may be nanoparticles. In this regard, the metal particles may have an average size (in terms of average longest dimension) of between 0.01 microns and 0.1 micron or between 0.001 micron and 1 micron; or no greater than 5 microns.

In embodiments that include an polymeric binder, the polymeric binder can include any suitable polymeric binder, for example, a polyurethane, a polyvinyl butyral, a polyacrylate, polyvinyl acetate, polystyrene, polystyrene acrylate, a polyurea, a polyimide, an amide, an epoxy, a glycidyl-Si-Zr-containing solgel, a polyester, a phenoxy resin, a polysulfide, or mixtures thereof.

In some embodiments, conductive polymer may be present in the electrical bridge 145 in an amount of at least 5 wt. %, at least 10 wt. %, at least 30 wt. %, at least 50 wt. %, or at least 90 wt. %, based on the total weight of the composite material that forms the electrical bridge 145.

In some embodiments, metal particles may be present in the electrical bridge 145 in an amount of at least 0.01 wt. %, at least 0.1 wt. %, at least 1.0 wt. %, at least 5 wt. %, or at least 20 wt. %, based on the total weight of the composite material that forms the electrical bridge 145. Generally, the amount of metal particles present in the electrical bridge may be that which is necessary to convert the conductive polymer from the first impedance state to the second impedance state acid state upon exposure to a sterilant.

In some embodiments, polymeric binder may be present in the electrical bridge 145 in an amount of at least 5 wt. %, at least 10 wt. %, at least 40 wt. %, at least 50 wt. %, or at least 90 wt. %, based on the total weight of the composite material that forms the electrical bridge 145.

In some embodiments, in addition to a change in impedance state, the electrical bridge 145 may additionally exhibit a change in color. For example, in embodiments in which the electrical bridge 145 includes PANI, the electrical bridge 145 may begin in a first impedance state having a first color (e.g., green) and a second impedance state having a second color (e.g., blue or yellow). In this manner, visual determination of the adequacy of a sterilization cycle may be carried out.

In some embodiments, the sensor device 130 may be a stand-alone device that can be placed into a sterilization system 100. In further embodiments, the sensor device 130 may be incorporated into another device (e.g., sterilization process challenge device with a torturous path such as porous matrix or a lumen channel, Bowie-Dick test pack, or the like) which may include a housing and one or more internal components or materials that are configured to facilitate assurance that adequate sterilization conditions are present during a sterilization cycle.

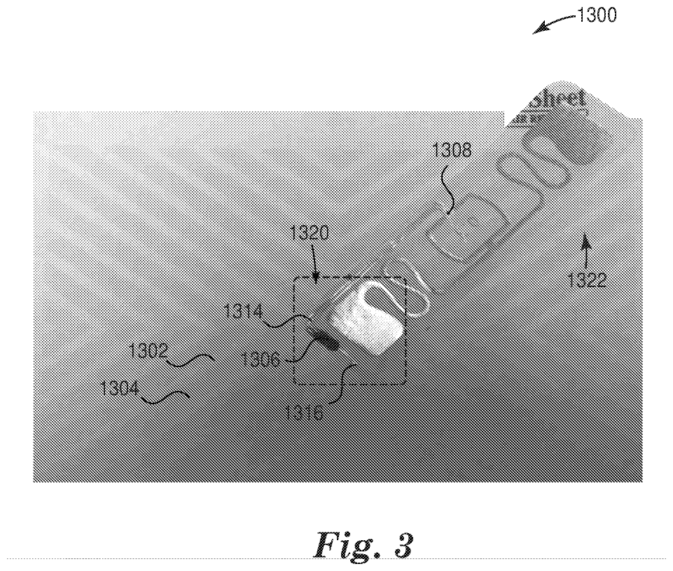

Referring now to FIG. 3, use of the sensor device 130 in sterilization system 100 in accordance with some embodiments of the present disclosure is illustrated. As shown, the sensor device 130 may be disposed within the chamber 110 of sterilization system 100. In some embodiments, the sensor device 130 may be disposed within the chamber 110 such that it may interact with the component(s) of the sterilant stream 120 upon entry into the chamber 110. In some embodiments, a reader device 160 may also be provided.

In some embodiments, the reader device 160 may be configured to receive signals from the sensor device 130 and translate the received signal into a determination that relates to the adequacy of a sterilization cycle (e.g., a pass/fail determination). For example, the reader device 160 may be configured to interrogate the sensor device 130 such that the reader device 160 measures the impedance across the electrode pair (e.g., individual readings or continuous or semi-continuous readings over time) which can correspond to whether various environmental conditions were or were not achieved in the sterilization process, or whether an adequate sterilization process was achieved. In some embodiments, when exposed to a first environmental condition (e.g., ambient conditions), the reader device 160 (if interrogating the sensor device) would measure a first impedance value that is indicative of whether the conductive polymer of the electrical bridge 145 is in a first impedance state or a second impedance state. As described above, an environmental condition change (or second environmental condition) within the chamber 110 can change the impedance state of the conductive polymer and, in turn, the impedance across the electrode pair measured by the reader device 160. In some embodiments, when the conductive polymer is in the first impedance state a first resistance is measurable across the first and second electrode, and when the conductive polymer is in the second impedance state a second resistance is measurable across the first and second electrode, and the first resistance is different than the second resistance.

In some embodiments, the reader device 160 may be in electronic communication (or capable of electronic communication) (continuously or at any desired interval) with the sensor device 130 (e.g., wireless communication such as Bluetooth, RF, or Near-Field communication, or wired communication via a suitable electronic connection (e.g., a pair of electrical leads that may be coupled to an electrode pair of the sensor device 130)). In some embodiments, the reader device 160 may be a device for measuring electrical resistance (e.g., an electrical multimeter).

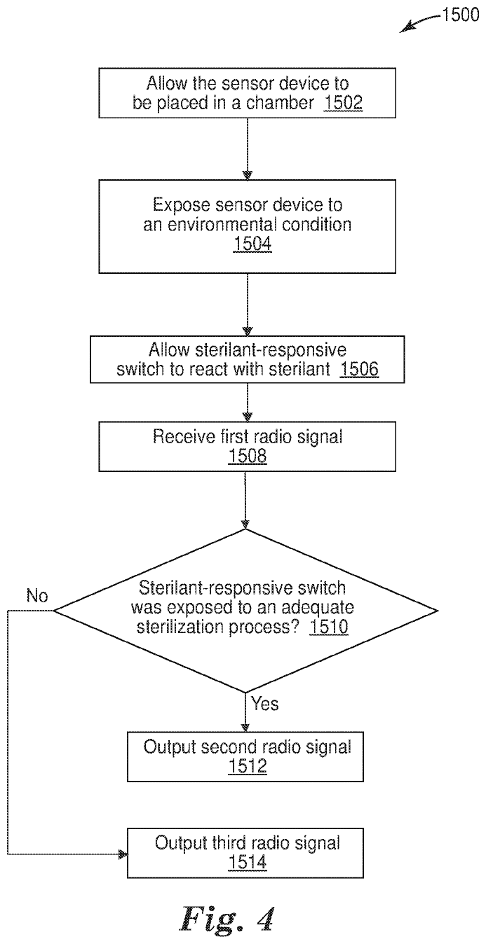

Referring now to FIG. 4, use of the sensor device 130 in a sterilization system 100 in accordance with some embodiments of the present disclosure is illustrated. As shown, the sensor device 130 may again be disposed within the chamber 110 of sterilization system 100 such that it may interact with the component(s) of the sterilant stream 120 upon entry into the chamber 110. Additionally, one or more medical devices 165 to be sterilized may be disposed with the chamber 110. For example, as shown, the sensor device 130 and the one or more medical devices 165 may be housed to together in a package 170 (often referred to in industry as a tray). It is to be appreciated that each package 170 may house any number of medical devices 165 or number of sensor devices 130. Alternatively, the sensor device 130 and the one or more medical devices 165 may be housed separately within the chamber 110. As shown, embodiments, a reader device 140 may also be provided.

In some embodiments, the present disclosure further relates to methods of using the sensor device 130 in a sterilization system 100. The method may begin with a user placing the sensor device 130 in the chamber 110. As previously discussed, the sensor device 130 may be placed alone in the chamber 110 or may be placed with one or more medical devices to be sterilized (and may be packaged in a tray with medical devices or disposed in the chamber 110 separate from the medical device or medical device tray). After the sensor device is placed in the chamber, the chamber 110 can be sealed from the environment.

In some embodiments, a user can then activate a sterilization process of the sterilizer and the sensor device can be exposed to a sterilant and/or one or more environmental conditions in a sterilization process. For example, if the sterilant is steam, then the sterilant may be at least 95% saturated steam/water vapor and the sterilization process may include achieving a temperature within the chamber 110 of at least 132 or at least 134 degrees Celsius for at least 2 minutes or at least 121 degrees Celsius for at least 8 minutes or at least 10 minutes. As an additional example, if the sterilant is hydrogen peroxide, then the sterilant may be in an atmosphere containing at least 30% hydrogen peroxide vapor and the sterilization process may be carried out at least 50 degrees Celsius for at least 60 minutes. Various standards for each sterilant can exist and may vary based on the manufacturer, article to be sterilized, or combinations thereof.

In some embodiments, as discussed above, exposing the sensor 130 to the sterilant and/or the conditions within the chamber 110, may result in a change of the impedance state of the conductive polymer of the electrical bridge 145.

In some embodiments, the method may further include continuously, intermittently, or at any desired time, the reader device 160 receiving signals from the sensor device 130 and translating such received signal into a determination that relates to the adequacy of a sterilization cycle (e.g., a pass/fail determination). As discussed above, the received signals may relate to a measured impedance across the electrode pair, which corresponds to various environmental conditions that were or were not achieved in the sterilization process. For example, a measured impedance above or below a predetermined value may be used to determine whether adequate sterilization process conditions were achieved within the chamber 110.

In some alternative embodiments, aspects of the present disclosure relate to a sensor device having a sterilant-responsive switch that is responsive to environmental conditions (including sterilant) in a sterilization process. The sterilant-responsive switch can be electrically coupled to conductive traces of the sensor device and can be mechanically activated or formed from a conductive polymer material.

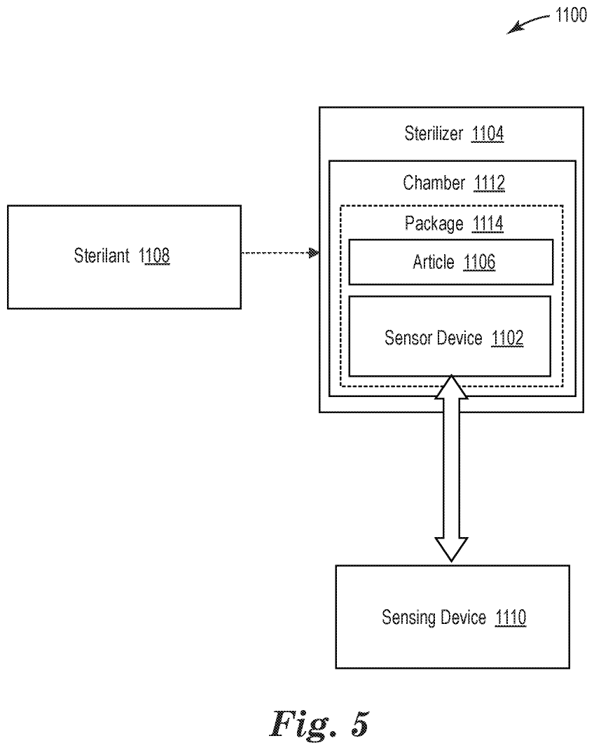

FIG. 5 illustrates a sterilization indicator system 1100. The sterilization indicator system 1100 can include a sterilizer 1104.

The sterilizer 104 is configured to provide a sterilant 1108 to a chamber 1112 in a sterilization process. Various examples of sterilizer 104 can exist and each sterilizer can differ as to the type of sterilant 1108 provided. Sterilizer 1104 can be based on steam, or hydrogen peroxide, for example, vaporized hydrogen peroxide, and each type can have different sterilization process conditions. Examples of sterilizers using hydrogen peroxide as a sterilant are commercially available from Steris (Mentor, OH) or Tuttnauer (Israel). Examples of sterilizers using steam as a sterilant are commercially available from Steris (Mentor, OH).

The chamber 1112 can have one or more environmental conditions. In at least one embodiment, the environmental condition can be related to conditions inside of the chamber 1112 and can include, but not limited to, exposure time, sterilant, temperature, pressure, or combinations thereof. For example, a first environmental condition can exist pre-sterilization process and a second environmental condition can exist during the sterilization process. A sensor device 1102 can determine whether the second environmental condition corresponds to an adequate sterilization process. An adequate sterilization process can vary based on the sterilant used, the manufacturer of the sterilizer, and the article 1106 to be sterilized. For example, Guideline for Disinfection and Sterilization in Healthcare Facilities, Center for Disease Control (2008) provides minimum cycle times for sterilization of various article 1106 types and sterilant 108 in Tables 1 and 7, which are incorporated by reference.

The sterilization indicator system 1100 includes a sensor device 1102 that is capable of collecting and providing data regarding the environmental conditions within chamber 1112 with respect to the sterilization process. Further, the sensor device 1102 can also be read by a sensing device 1110. The sensing device 1110 is an electronic device that can read the environmental conditions remotely. In one example, the sensing device 1110 can read the sensor device 1102 to determine environmental conditions in the chamber 1112 in real-time through the walls of the chamber 1112. For example, a wall can have a hole formed therein for directly reading an RFID tag through the steel wall. In another example, the sensing device 110 can read/interrogate the sensor device 1102 to determine environmental conditions of the chamber 112 when outside of the walls of the chamber 1112, e.g., when in a wrapped package 1114. In at least one embodiment, an adequate sterilization process can change the electrical impedance of the sensor device 1102 and be detected by the sensing device 1110.

The sensing device 1110 can use wireless communication or wired communication to read the sensor device 1102. For example, if wired, then the sensor device 1102 can include a memory element to store the environmental conditions captured by the sensor device 1102. In at least one embodiment, the sensor device 1102 can be affected by past environmental conditions and be chemically or electrically modified. For example, the sensor device 1102 can also include a sterilant-responsive switch that indicates, directly or indirectly, the environmental condition from the sterilization process in the chamber 1112.

The sensor device 1102 can include any type of sterilant-resistant integrated circuit or simple open circuit. The sensor device 1102 can include any appropriate electrical connection to communicate with a sensing device 1110 that detects and measures any electrical signals generated. Such connections may include, but are not limited to, hard wiring, physical electrical contacts, e.g., spring-loaded or jacks, Ethernet, Bluetooth, 802.11, wireless local area networks (WLANs), WiFi, WiMax and the like, or any other wired or wireless communication type known in the art.

For example, the sensor device can be an RFID tag, a thermometer, a pressure sensor, a communication device, or combinations thereof. In at least one embodiment, the sensor device 1102 is an RFID tag and the sensing device 1110 is an RFID interrogator device. Example RFID interrogator devices can be based on UHF and commercially available from Zebra (Lincolnshire, IL), Alien Technology (San Jose, CA), or Impinj (Seattle, WA). “Other example RFID interrogator device can also be based on High Frequency (HF) and commercially available from Jadak (Syracuse, NY), Technology Solutions Ltd (United Kingdom), Samsung, or Apple or be based on Low Frequency (LF) and commercially available from RFID Inc. (Aurora, CO), Gao RFID Inc. (Ontario, Canada), or SkyRFID Inc. (Ontario, Canada).”

The sensor device 102 can be paired with one or more components such as a substrate and environmental change receptor to form a sterilization indicator sensor which is described further herein. In at least one embodiment, the environmental change receptor is distinct from the sterilant-responsive switch. For example, the environmental change receptor can be configured to affect the admittance/impedance of the sterilant-responsive switch.

In at least one embodiment, the article 1106 and sensor device 1102 can be wrapped in a package 1114. The sensor device 1102 can be responsive to the sterilization process occurring in the chamber 1112. The sensor device 1102 can be read as to determine whether the using the sensing device 1110 without unwrapping the package 1114 which helps assure sterility of the article 1106 to an end user.

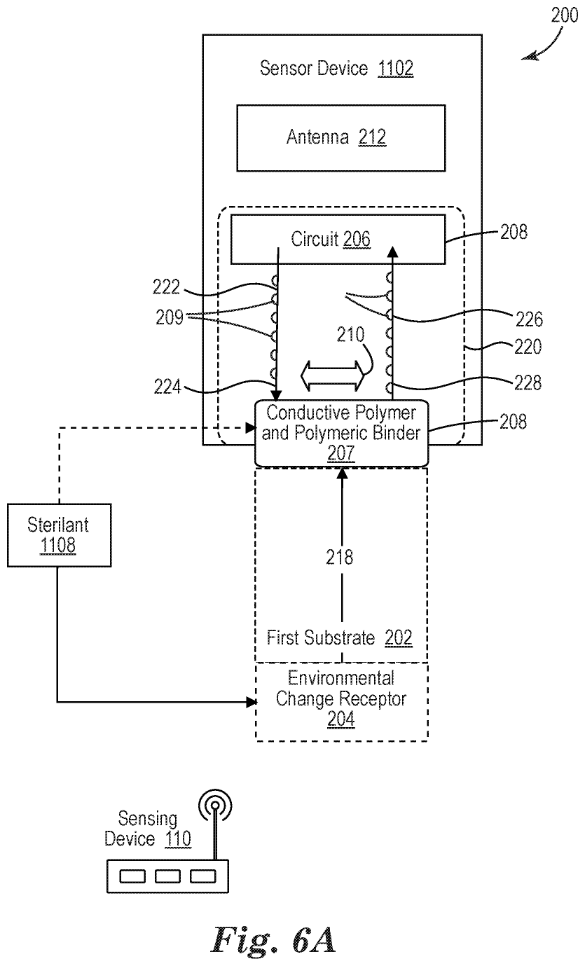

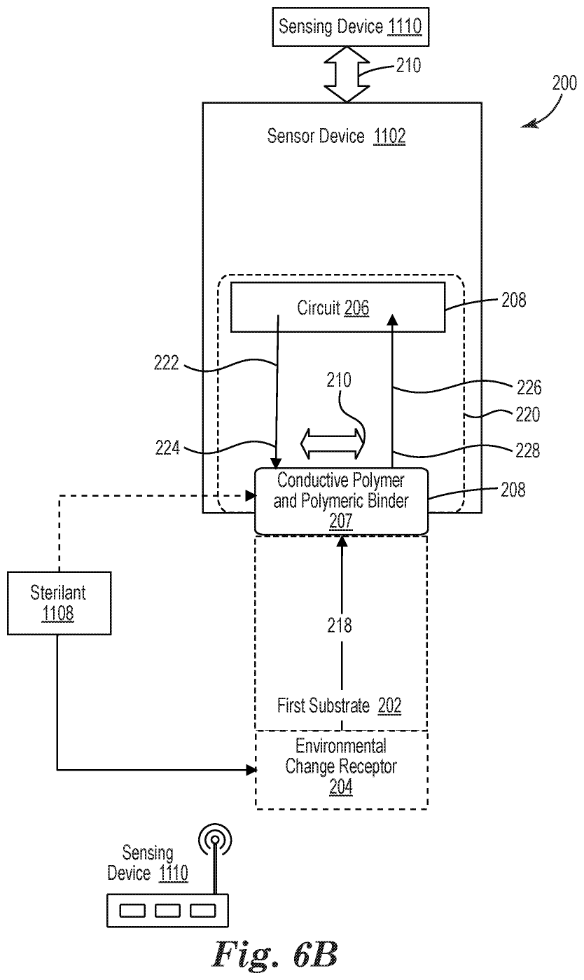

FIG. 6A illustrates a sterilization indicator sensor 200 for use in the sterilizer.

The sterilization indicator sensor 200 can include the sensor device 102 described herein. In at least one embodiment, the sensor device 102 can include a monitoring loop 220. The monitoring loop 220 can include the sterilant-responsive switch 208 which is electrically modifiable based on exposure to environmental conditions for the sterilization process, particularly an adequate sterilization process. In at least one embodiment, the monitoring loop 220 is configured to electrically change based on exposure to an adequate sterilization process. For example, the monitoring loop 220 can increase or decrease in admittance/impedance based on exposure to an adequate sterilization process.

The sterilant-responsive switch 208 can be based on a conductive polymer material or mechanical interaction with various components such as an environmental change receptor 204. In at least one embodiment, the sterilant-responsive switch 208 can include a circuit 206, a conductive polymer having a first state and a second state, and a polymeric binder (collectively, 207). In at least one embodiment, the sterilant-responsive switch 208 can be binary. For example, the sterilant-responsive switch 208 can be triggered from off to on indirectly based on interaction of the sterilant with environmental change receptor 204. In at least one embodiment, the circuit 206 can be an integrated circuit.

The sterilant-responsive switch 208 can also have a graduated response to the environmental condition. For example, a conductive polymer material may suffer from gradual electrical admittance degradation based on interaction from a sterilant 108. Examples of sterilant-responsive switch 208 are described further herein.

A conductive polymer material can be any substance that has semi-conductive properties or that is switchable between a first state and a second state. In other words, the conductive polymer is capable of being converted from being in the first state to being in the second state when in contact with a sterilant in at least one embodiment, the first state can be a first impedance state having a first impedance and the second state can be a second impedance state having a second impedance, for example, a solid substance that has conductivity between that of an insulator and a metal. In at least one embodiment, the impedance state can be related to the impedance and the admittance of the sensor device. The impedance state can be related to an opposition to flow of the conductive polymer material and include aggregation of its resistance, and inductive and capacitive reactances. In at least one embodiment, the first state can be a non-conductive state and the second state can be a conductive state and vice versa. The conductive state can be a doped conductive state and the non-conductive state can be a non-conductive reduced form or a non-conductive oxidized form of the conductive polymer. In at least one embodiment, the conductive polymers may be in forms of conductive polymer electrolytes, for example, protonated forms. In at least one embodiment, the sterilant-responsive switch connects the circuit in the first state and disconnects the circuit in the second state.

The conductive polymer material can include an electrically active polymer that changes from a first impedance state to a second impedance state or a second impedance state to a first impedance state based on interactions with an environmental change receptor 204, an environmental condition, a conductive trace, or combinations thereof. In at least one embodiment, the first impedance state can either correspond to having higher or lower impedance relative to the second impedance state depending on the mechanism. For example, polyaniline can switch from non-conductive to conductive or vice versa. In at least one embodiment, the first impedance state refers to having an admittance and impedance sufficient to electrically bridge an open circuit, e.g., having an admittance of at least 2 siemens. The electrically active polymer can be a semi-flexible rod polymer. In at least one embodiment, the electrically active polymer can have a repeat unit of: aniline, acetylene, pyrrole, phenylene, phenylene vinylene, phenylene ethynylene, phenylene sulfide, fluorene, pyrene, azulene, napthalene, carbazole, indol, thiophene, ethylene dioxythiophene, or combinations thereof. The electrically active polymer can be doped or undoped with various dopants such as dinonylnaphthalene sulfonic acid (DNNSA), sodium, arsenic pentafluoride, triiodide, camphorsulfonate, methanesulfonic acid, halogens or polyhalogen ions, methanol, hydrogen sulfate, hydrochloric acid, tetrafluoroborate, sodium sulfite, or combinations thereof. Preferably, the conductive polymer material is polyaniline (PANI) which can be in one of three oxidation states (leucoemeraldine, emeraldine (in the salt or base forms), and per(nigraniline). The emeraldine can be non-conductive in the base form and conductive in the salt form. Further, the emeraldine salt can be converted into the leucoemeraldine salt or per(nigraniline) which are non-conductive, via a reduction reaction, when sterilant-responsive switch 208 in contact with steam or hydrogen peroxide. The conductive polymer can be converted to non-conductive polymer via a de-doping reaction, when sterilant-responsive switch 208 in contact with steam or hydrogen peroxide

The polymeric binder can include any suitable binder, for example, a polyurethane, a polyvinyl butyral, a polyacrylate, polyvinyl acetate, polystyrene, polystyrene acrylate, a polyurea, a polyimide, an amide, an epoxy, a glycidyl-Si-Zr-containing solgel, a polyester, a phenoxy resin, a polysulfide, or mixtures thereof. With a polymeric binder, the reduced non-conductive PANI can maintain the non-conductive state much longer without going back (at least a year) in certain constructions such as an aluminum electrode pair or certain threshold of certain metal particles such as tin nanoparticles above 2% w/w in the formulation It was found surprisingly without a binder, the conductive polymer material, can change from a first state to a second state, but the second state without a binder is reversable. For example, PANI can be reduced through the steam sterilization but the redox state of PANI without a binder is reversable, i.e. the non-conductive PANI can reverse back to the conductive PANI form quickly in the air. In addition, the conductive polymer, for example, PANI, can stick on metal surface but once it goes through the sterilization process, the reduced non-conductive PANI can be easily delaminated from the metal surface (chip away). In addition, when polymeric binder is used, the conductivity of PANI in a solid film can be significantly increased without an alcoholic wash. Thus, the alcoholic wash is optional when a binder is present and users can save cost and time without the additional step of alcoholic wash.

Further, the sensing device 1110 can be configured to interrogate the sensor device 1102 such that the sensor device 1102 provides a plurality of impedance states over time which can correspond to various environmental conditions in the sterilization process. For example, the sensor device 1102, when exposed to a first environmental condition, can transmit a first impedance state based on the interaction (direct or indirect) of the sterilant-responsive switch with the first environmental condition. An environmental condition can change the measured capacitance of the sterilant-responsive switch 208. The sensor device 1102, when exposed to a second environmental condition, can transmit a second impedance state based on the interaction (direct or indirect) of the sterilant-responsive switch with the second environmental condition, and so forth with a third impedance state and a fourth impedance state. In at least one embodiment, the sensing device 1110 can determine the environmental conditions based on the impedance states and provide a graduated view of the environmental conditions over time (as opposed to a binary pass/fail that may be present).

The sensor device 1102 can include a first electrode 214 having a first end 222 and a second end 224 and a second electrode 216 having first end 226 and second end 228. The first ends of both electrode 214 and electrode 216 are electrically coupled to the circuit 206. In at least one embodiment, the second ends of electrode 214 and electrode 216 are not integrally attached using the same material as that of electrode 214 or electrode 216. In at least one embodiment, the second ends of electrode 214 and electrode 216 can each be connected through a sterilant-responsive switch 208.

In at least one embodiment, the distance 210 between electrode 214 and electrode 216 as measured along a sterilant-responsive switch 208. The distance 210 can be sufficient to sense a change in the electrical admittance/impedance without causing electrical shorts or interference between the electrode 214 and electrode 216. For example, if the distance 210 is zero, then electrode 214 and electrode 216 would be electrically coupled regardless of changes in the sterilant-responsive switch 208 and the monitoring loop 220 would not sense the environmental condition.

The electrode can include a metal, metal particle, carbon black, graphene, conducting polymers, carbon nanotubes or combinations thereof. In at least one embodiment, the oxidation potential of the metal is greater than the reduction potential of the conductive polymer. Example if suitable metal can include aluminum, iron, zinc, tungsten, molybdenum, tin, nickel, copper, or alloys thereof. For example, the use of aluminum has been surprisingly found to directly react with PANI and convert emeraldine salt into leucoemeraldine salt. The monitoring loop 220 can thus turn from a first impedance state to a second impedance state based on the redox reaction of the conductive polymer material with the metal at the environmental condition corresponding to an adequate sterilization process (e.g., of steam).

In at least one embodiment, the sterilant-responsive switch 208 include a conductive particle 209. In some embodiments, the conductive particle 209 can be coated on the first electrode and the second electrode, as shown in FIG. 6A. In at least one embodiment, the conductive particle can include a metal containing particle and the metal of the metal containing particle is selected from copper, cobalt, manganese, zinc, iron, silver, tin, lead, gallium, platinum, osmium, iridium palladium, ruthenium, rhodium, gold, or alloy thereof. In at least one embodiment, the conductive particle comprises active carbons, C60, carbon nanotubes, graphite, metal oxides, and conductive organic polymeric particles comprising insoluble conducting polymers, such as polyaniline, polypyrrole, and polythiophene, or combinations of conductive inorganic particles and organic conductive particles.

In at least one embodiment, the sterilization indicator sensor 200 can include only the sensor device 1102. The sterilization indicator sensor 200 can also optionally include the first substrate 202 and/or the environmental change receptor 204.

In at least one embodiment, a portion of the sterilant-responsive switch 208 can contact the first substrate 202. The first substrate 202 can be either wicking or non-wicking. If non-wicking, the first substrate 202 can be any metallic layer such as aluminum foil, or polymeric layer such as polyethylene, polyurethane, or polyester layer. In at least one embodiment, the first substrate 202 can provide structural support to the sensor device 102. The first substrate 202 can also provide support to the environmental change receptor 204.

If wicking, the first substrate 202 can be any suitable material through which the organic compound can migrate by capillary action. The preferred wicking first substrate 202 is a paper strip. Other such wicking materials such as non-woven polymeric fabrics and inorganic fibrous compositions may be used. The dimensions of the wicking first substrate 202 is not critical. However, its dimensions (thickness and width) will affect the rate of wicking and determine the quantity of organic compound required to result in a suitable scale length. Hence, from an economic standpoint the wicking first substrate 202 should be as thin as practical. A suitable width for the first substrate 202 is about 3/16 to about ¼ of an inch. Examples of the wicking first substrate 202 are Whatman No. 1 filter paper, Whatman No. 114 filter paper, supported microcrystalline cellulose (TLC plate), supported aluminum oxide, and supported silica gel. In some embodiments, the conductive particle 209 can be coated on the first substrate 202. In some embodiments, the first electrode and second electrode can be printed on the conductive particle coating.

In at least one embodiment, the environmental change receptor 204 is disposed proximate the first substrate 202. For example, the environmental change receptor 204 can be positioned such that the environmental change receptor 204 flows onto the first substrate 202 and is wicked from a first substrate position to a second substrate position (which may correspond to a portion of the sterilant-responsive switch 208) as indicated by flow direction 218. In at least one embodiment, the environmental change receptor 204 can also be disposed directly on the first substrate 202 at the first substrate position. In at least one embodiment, the environmental change receptor 204 is disposed proximate or adjacent to the sterilant-responsive switch 208. In at least one embodiment, the environmental change receptor 204 is solid and can be in the form of a tablet and disposed outside of the first substrate 202. In at least one embodiment, the environmental change receptor 204 can be embedded within or layered upon the first substrate 202.

The environmental change receptor 204 can include one or more environmentally responsive or sensitive materials selected depending on the sensing needs. The environmentally responsive material can be selected based on its solubility, boiling point, melting point, ability to absorb gases or liquids, softening point or flow properties, such that it changes properties (evaporates or redistributes on the sensor strip) in response to specific environmental conditions. In some cases, the environmental change receptor 204 can include more than one part, where each part can include similar or different environmentally responsive materials and be disposed at different locations. In at least one embodiment, the environmental change receptor 204 can be selected based on an ability to change the admittance/impedance of the sterilant-responsive switch. The environmental change receptor 204 can be acidic or basic to affect the first impedance state of a conductive polymer material. For example, if the environmental change receptor 204 is basic, then the base can react with emeraldine salt to form emeraldine base and change from a first impedance state to a second impedance state.

The environmental change receptor 204 can include a type of meltable or flowable material, for example, crystalline or semi-crystalline materials (e.g., Tetra-n-butylammonium bromide (TBAB)), thermoplastics, polymers, wax, organic compounds such as salicylamide, polyethylene-co-acrylic acid, sucrose and the like. In some cases, the environmentally responsive material is selected based on its response to combined conditions of temperature and humidity, or temperature, humidity and time. The material can be selected to tailor to a particular application. In some embodiments to monitor the presence of chemical substance, the environmental change receptor 204 can include a type of material absorbing or reacting with the chemical substance. In an example of detecting gas, the environmental change receptor 204 can include Zeolite HiSiv 3000 powder from UOP LLC, Des Plaines, IL.

Some environmental change receptors can be responsive to a steam sterilant in environmental conditions for an adequate sterilization process. In at least one embodiment, the environmental change receptor 204 can include an organic base having a melting point of greater than 100 degrees C. and miscible with salicylamide. For example, the organic base can be N, N-dimethylpyridine, adamantylamine, or combinations thereof.

Some environmental change receptors can also be responsive to a steam or hydrogen peroxide sterilant in an adequate sterilization process. Such an environmental change receptor can include various pigments and inks such as a blue colored ink and a pink pigment. Further the environmental change receptor can include an organic ester that is solid at room temperature. In at least one embodiment, the sterilant 108 can interact with environmental change receptor 204, sterilant-responsive switch 208, or both to produce a change which would affect sensor device 102.

The sensor device 200 can include an antenna 212 which is capable of receiving energy from and transmitting data to a sensing device 1110. Antenna 212 can be various shapes that are optimized for transmission to the sensing device 110. One example of an antenna 212 design is commercially available from Smartrac (Netherlands) under the Model name BELT.

In at least one embodiment, the antenna 212 can be formed such that it is unaffected by the sterilization process. For example, the antenna 212 can have no breaks within an antenna loop (but the sensor device 1102 may have a break within the monitoring loop 220). The antenna 212 can be electrically coupled to the integrated circuit 206 and form the antenna loop. The integrated circuit 206 can harvest energy from the sensing device 110 to transmit the antenna 212 impedance. Various integrated circuit 206 devices can be designed for RFID applications, such as passive, semi-active, and active RFID applications, and commercially available from NXP Semiconductors (Netherlands), Impinj (Seattle, WA), or Axzon (Austin, TX). An example of the integrated circuit 206 is under the trade designation Magnus from Axzon (Austin, TX) or the UCODE G2iM or G2iL+ from NXP Semiconductors which can include UHF RFID transponder capability and a tag tamper alarm capable of measuring the state of the monitoring loop 220.

In at least one embodiment, the sensor device 200 can include a second integrated circuit responsive to a different frequency than the first integrated circuit. The second integrated circuit can be electrically coupled to the antenna 212 or a second antenna. The second integrated circuit can also be electrically coupled to the monitoring loop.

FIG. 6B illustrate a sterilization indicator sensor 200 that is similar to sterilization indicator sensor 200 of FIG. 6A except that the circuit 206 is read through a direct physical contact with the sensing device 1110 for impedance or resistance measurement. The direct physical contact can be a hard-wired electrical connections 250 between the electrodes and the electrical circuit used to detect and measure the electrical signal resulting from electron transfer.

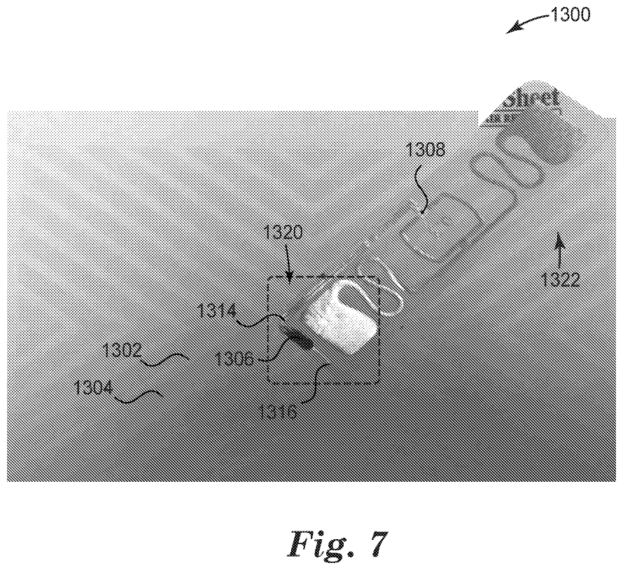

FIG. 7 illustrates a sterilization indicator sensor 1300 at a different view. The conductive trace 1314 and conductive trace 1316 are shown contacting the polymeric gate material 1306. Once exposed to a sterilant, the polymeric gate material 1306 can change admittance/impedance which is sensed by an RFID interrogator device.

In at least one embodiment, the sterilization indicator sensor 1300 can be present in a stack of cards which can generally be paper or formed from the first substrate. The sterilization indicator sensor 1300 can be structurally similar to the chemical indicator described in U.S. Pat. No. 9,170,245 which is incorporated by reference. In at least one embodiment, the stack of cards can have the sterilization indicator sensor 1300 positioned medially in the stack of cards.

In at least one embodiment, the sterilization indicator sensor 1300 can form a central zone 1320 and a peripheral zone 1322. Peripheral zone 1322 can surround a central zone 1320. In at least one embodiment, the central zone 1320 can have only partial contact with the sterilant occurred when placed in the stack of cards. The central zone 1320 can be a result of an air pocket formed by the stack of cards with sterilization indicator sensor 1300. In at least one embodiment, the central zone can mirror the shape of the sterilization indicator sensor 1300. For example, the central zone 1320 can be a rectangular (such as a rhomboid), or elliptical shape. In one example, the sterilization indicator sensor 1300 has an area of no greater than 25 square inches and a central zone 1320 of no greater than 1 square inch. Thus, the ratio of overall area to the central zone area can be no greater than 25:1.

In at least one embodiment, the air pocket can be representative of a challenge path that is sterilized last. In at least one embodiment, the polymeric gate material 1306 is positioned in the geometric center of the first substrate 1302 and/or the central zone such that the polymeric gate material 1306 detects whether an adequate environmental condition occurs in the central zone. For example, sterilant can interact with the peripheral zone 1322 but may take time to interact with the central zone 1322 when packaged in the stack of cards. As shown, the polymeric gate material 1306 contacts the ionic salt 1304.

In at least one embodiment, the stack can be completely wrapped in a sheet of material to form a wrapped package. For example, the sheet of material can be a nonwoven that can be a sterilant-permeable medical wrapping commercially available as a sterilization wrap.

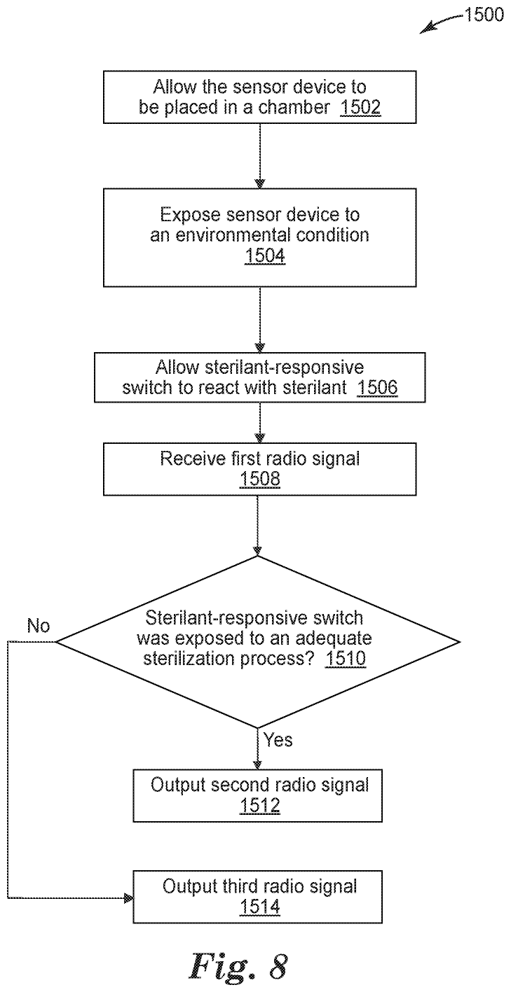

FIG. 8 illustrates a method 1500 of using the sensor device.

The method 1500 can begin at block 1502. In block 1502, a user can place the sensor device in the chamber of a sterilizer. In at least one embodiment, the user can place the sensor device with an article to be sterilized in the chamber. The user can also package the sensor device and the article in a wrapped package such that the sensor device is not visible while the package is wrapped. The sensor device is described further herein and includes a sterilant-responsive switch. In at least one embodiment, the user can place the sensor device can be a part of a sterilization indicator sensor which can be placed in the chamber. After the sensor device is placed in the chamber, then the chamber can be sealed from the environment.

In block 1504, a user can activate a sterilization process of the sterilizer and the sensor device can be exposed to a sterilant and/or one or more environmental conditions in a sterilization process. For example, if the sterilant is steam, then the sterilant is at least 95% saturated steam/water vapor and the sterilization process is 134 degrees Celsius for 2 minutes or 121 degrees Celsius for 10 minutes. In another example, if the sterilant is hydrogen peroxide, then the environmental condition is an atmosphere containing 31% hydrogen peroxide vapor and the sterilization process is 50 degrees C. for 60 minutes. Various standards for each sterilant can exist and may vary based on the manufacturer, article to be sterilized, or combinations thereof. In at least one embodiment, the environmental condition includes the presence of the sterilant.

In block 1506, the sterilant-responsive switch of the sensor device or the sterilization indicator sensor can react with the sterilant or react (physically or chemically) with the environmental condition (which can include the sterilant). In at least one embodiment, the sterilant-responsive switch can also interact with a substrate or an environmental change receptor to modify the admittance/impedance of the sterilant-responsive switch. For example, the environmental condition, environmental change receptor, or combinations thereof, can cause sterilant-responsive switch to change from the first state to the second state, for example, from a first impedance state to a second impedance state, or vice versa. In at least one embodiment, upon exposure to an adequate environmental condition comprising a steam sterilant or hydrogen peroxide sterilant, the conductive particle can react with the sterilant and/or conductive polymer to change impedance of the conductive polymer. In at least one embodiment, upon exposure to an adequate environmental condition comprising a steam sterilant or hydrogen peroxide sterilant, the conductive particle can react with the sterilant first and subsequently react with the conducting polymer to change the impedance of the conductive polymer. In at least one embodiment, upon exposure to an adequate environmental condition comprising a steam sterilant or hydrogen peroxide sterilant, which can trigger a redox reaction in the sterilant-responsive matrix to change impedance of the conductive polymer.

In block 1508 through block 1514, a sensing device can be configured to read the sensor device to determine whether the first impedance state is present.

In at least one embodiment, the sensing device is configured to read the sensor device through a wrapped package. The sensing device can also be configured to read the sensor device when the chamber is sealed (i.e., through a housing of the sterilizer). The sensing device can use an onboard memory to later read the sensor device. In at least one embodiment, the sensing device can be an RFID interrogator device. The sensing device can be configured to transmit a first radio signal to the sensing device in block 1508. The first radio signal can be a variety of frequencies but is preferably UHF (300 MHz-3000 MHz).

The first radio signal can affect the sensor device and the sensor device can emit a second radio signal or a third radio signal in block 1512, or block 1514. For example, in decision block 1510, if the sterilant-responsive switch was exposed to a sterilization process, for example, an adequate sterilization process, then the sensor device can output a second radio signal in block 1512. If the sensor device was not exposed to an adequate sterilization process, then the sensor device can output a third radio signal in block 1514. In at least one embodiment, the output can be inherent and not require any computational resources of the sensor device. In at least one embodiment, the second radio signal can be indicative of whether the sterilant-responsive switch has degraded (e.g., the sterilant cause degradation of the sterilant-responsive switch directly or indirectly). In at least one embodiment, the second radio signal can be indicative of whether the sterilant-responsive switch completed a circuit of a monitoring loop of the sensor device. The third radio signal can be indicative of no degradation or minimal degradation of the sterilant-responsive switch.

The presence of the second or third radio signal can indicate to the sensing device whether the sensor device was exposed to environmental conditions from an adequate sterilization process. The sensing device can further communicate whether the adequate sterilization process was achieved and perform subsequent actions as a result.

### EXAMPLES

These examples are merely for illustrative purposes only and are not meant to be limiting on the scope of the appended claims. All parts, percentages, ratios, etc. in the examples and the rest of the specification are by weight, unless noted otherwise. Materials used in the Examples and their sources are provided in Table 1. Solvents and other reagents used were obtained from MilliporeSigma, St. Louis, MO, unless otherwise noted.

TABLE 1
Materials

| Material | Source (location) |
|---|---|
| Modified Tamper evident | Smartrac (Irvine, CA) |
| RFID Inlays | |
| Polyaniline | Boron Molecular (Raleigh, |
| | North Carolina) |
| Desmodur N3390 | Covestro (Pittsburgh, PA) |
| CAPA 3031 | Perstorp (Toledo, OH) |
| Zoldine MS-plus | Angus Chemical Company |
| | (Chicago, IL) |
| ThingMagic RFID Reader | Jadak (Syracuse, NY) |
| Comply Bowie-Dick | 3M Company (St Paul, MN) |
| Test Pack | |
| Electric Multimeter, 89IV | Fluke Corporation (Everett, WA) |
| Tin nanopowder | US Research Nanomaterials |
| (60-80 nm) | (Houston, TX) |
| Silver nanopowder | US Research Nanomaterials |
| (50-80 nm) | (Houston, TX) |
| Copper nanopowder | US Research Nanomaterials |
| (40 nm) | (Houston, TX) |
| X-rite Densitometer | X-rite (Grand Rapids, MI) |
| AMSCO Lab 110 | Steris (Mentor, OH) |
| Sterrad 100S | American Sterilization Products |
| | (Irvine, CA) |
| Getinge steam sterilizer | Getinge (Gothenburg, Sweden) |

### Experiment 1

Four grams of the parent solution prepared according to the formulation provided in Table 2 was aliquoted to 4 different vials. To the first vial, 160 mg of silver nanopowder was added. To the second vial, 80 mg of tin nanopowder was added. To the third vial, 80 mg of copper nanopowder was added. The fourth vial served as a control. Each vial was then sonicated for 10 minutes. A fourth vial without any added metal powder was used as a control. Each of the nanopowder solutions was then sequentially diluted in different vials by mixing equal portions of the nanopowder solution and the parent solution (no metal) to generate PANI/metal solutions from 4% to 0.25% (weight percentages based on parent formulation). All solutions were mixed well with a small stirring bar for one hour.

Samples of each of these solutions were then coated on 3-mil-thick poly(ethylene terephthalate) (PET) film using a #24 Meyer bar and put in an oven at 145° C. for 10 min. The coated PET samples were then cut into about 1-inch wide strips and attached to a white Nylon membrane sheet. Color was measured with an X-rite densitometer. Resistance was measured using a multimeter with the two pins 1 inch apart. After color and resistance measurements, one sheet was subjected to a steam sterilization cycle (Getinge) at 134° C. for 4 minutes. Another sheet was sterilized using a hydrogen peroxide sterilizer (Sterrad 100S). After each sheet was processed, the samples on each sheet were measured again with both X-rite and multimeter.

Tables 3 and 4 show the color changes and resistance changes before and after sterilization with steam and hydrogen peroxide, respectively. From Table 3, one can see that Sn nanopowder samples showed significant resistance changes and a significant increase of a* value (color changed from green to light yellow) after steam sterilization. This indicates that tin nanopowder can effectively convert conductive PANI to a non-conductive form. The samples made with silver and copper nanopowders did not cause significant electric resistance changes when the coated sensors were exposed to the same steam sterilization cycle. In Table 4, we can see that all copper solutions from 2% to 0.25% and a relatively high concentration of silver nanopowder (4%) can effectively change the coating resistance when the sensors were exposed to a hydrogen peroxide-based sterilization cycle.

It was noted that b* values also changed significantly (from green to dark emerald green). It is hypothesized that both copper and silver can serve as catalysts to degrade hydrogen peroxide to generate a hydroxide ion which neutralizes the acid-doped PANI to convert it from conductive to non-conductive form (a de-doping process).

TABLE 2
Parent formulation for making metallic
nanopowder doped solutions

| Component | % (weight base) |
|---|---|
| 50% PANI in toluene | 14.92 |
| MIBK | 41.98 |
| Xylene | 29.57 |
| Desmodur 3390 | 8.48 |
| CAPA 3031 | 4.75 |
| Zoldine MS-plus | 0.3 |

TABLE 3
Colors and resistances of coated film samples before
and after steam sterilization at 134° C. for 4 minutes

| | Before steam | | | | After Steam | | | |
|---|---|---|---|---|---|---|---|---|
| | | | | Resistance | | | | Resistance |
| Sample | L* | a* | b* | (Mohm) | L* | a* | b* | (Mohm) |
| 4% Ag | 41.71 | −21.16 | 33.57 | 2 | 41.96 | −20.86 | 32.46 | 1.1 |
| 2% Ag | 59.56 | −23.24 | 52.44 | 0.85 | 59.43 | −22.28 | 48 | 0.05 |
| 1% Ag | 61.25 | −24.05 | 54.36 | 0.6 | 60.63 | −22.69 | 50.21 | 0.2 |
| 0.5% Ag | 60.84 | −23.49 | 53.95 | 0.59 | 62.84 | −23.11 | 53.02 | 0.35 |
| No Metal | 64.23 | −23.13 | 51.38 | 1.2 | 66.09 | −22.16 | 52.78 | 0.56 |
| control | | | | | | | | |
| 2% Cu | 55.72 | −22.4 | 49.13 | 0.17 | 56.68 | −20.78 | 46.3 | 0.05 |
| 1% Cu | 52.92 | −23.77 | 43.02 | 1.35 | 50.4 | −25.93 | 38.18 | 0.42 |
| 0.5% Cu | 52.93 | −24.19 | 42.87 | 1.44 | 51.24 | −26.77 | 38.57 | 0.4 |
| 0.25% Cu | 52.88 | −23.19 | 42.85 | 1.21 | 52.44 | −28.34 | 39.56 | 0.43 |
| 2% Sn | 49.15 | −14.56 | 41.22 | 0.88 | 58.01 | −3.56 | 16.92 | >60 |
| 1% Sn | 62.35 | −19.35 | 50.74 | 0.75 | 69.82 | −5.6 | 21.82 | >60 |
| 0.5% Sn | 62.42 | −20.51 | 50.73 | 1.2 | 78.86 | −8.39 | 31.75 | >60 |
| 0.25% Sn | 63.98 | −21.6 | 51.07 | 1.05 | 78.79 | −10.68 | 43.32 | >60 |

TABLE 4
Colors and resistances of coated film samples before
and after hydrogen peroxide sterilization

| | Before H2O2 | | | | After H2O2 | | | |
|---|---|---|---|---|---|---|---|---|
| | | | | Resistance | | | | Resistance |
| Sample | L* | a* | b* | (Mohm) | L* | a* | b* | (Mohm) |
| 4% Ag | 44.12 | −21.14 | 34.42 | 2.35 | 33.09 | −22.99 | 15.42 | >60 |
| 2% Ag | 60.42 | −23.15 | 52.95 | 0.2 | 53.91 | −28.57 | 39.46 | 1.4 |
| 1% Ag | 60.56 | −24.15 | 54.2 | 0.36 | 53.18 | −30.11 | 40.08 | 1.45 |
| 0.5% Ag | 62.63 | −23.65 | 54.79 | 0.54 | 57.04 | −29.41 | 40.2 | 1.35 |
| No Metal | 65.62 | −23.21 | 51.94 | 1.2 | 59.93 | −28.25 | 39.83 | 4.5 |
| control | | | | | | | | |
| 2% Cu | 55.68 | −22.3 | 49.3 | 0.2 | 48.04 | −27.62 | 34.54 | 1.8 |
| 1% Cu | 55.89 | −23.6 | 43.7 | 1.1 | 44.47 | −28.47 | 20.28 | 29.8 |
| 0.5% Cu | 53.8 | −24.61 | 43.48 | 0.99 | 41.49 | −29.47 | 19.75 | 37 |
| 0.25% Cu | 54.75 | −25.7 | 43.94 | 0.9 | 41.48 | −30.37 | 19.61 | 37 |
| 2% Sn | 47.99 | −14.14 | 40.19 | 0.8 | 43.9 | −17.39 | 32.01 | 1.9 |
| 1% Sn | 62.4 | −19.15 | 50.35 | 0.63 | 58.74 | −22.52 | 41.1 | 1.4 |
| 0.5% Sn | 64.03 | −20.66 | 51.2 | 0.91 | 58.7 | −25.27 | 39.41 | 2.8 |
| 0.25% Sn | 63.19 | −21.63 | 50.87 | 0.76 | 59.82 | −27.7 | 41.57 | 3.6 |

### Experiment 2

The procedure of Example 1 was used to prepare PANI/PU coating solutions containing 1% and 0.5% of copper and tin nanopowder, respectively. In a separate vial, the solution containing 1% tin and the solution containing 1% copper coating solution were combined in equal amounts and mixed well. The obtained samples were coated on 3 mil PET and tested as described in Example 1. Samples were sterilized with steam (AMSCO Lab 110, 134° C. for 3.5 minutes) and hydrogen peroxide (Sterrad 100S). Tables S5 and 6 show the electric resistance and color measurement changes. The results show that Sn is particularly responsive to steam sterilization while copper is reactive with hydrogen peroxide. Unexpectedly, the mixed solution containing both tin and copper offset the sensitivity of tin to steam sterilization while it did not affect the performance of copper in hydrogen peroxide sterilization.

TABLE 5
Color and resistance change from steam sterilization at 134° C. for 3.5 minutes

| | Before | | | | After | | | |
|---|---|---|---|---|---|---|---|---|
| | | | | R | | | | R |
| Sample | L* | a* | b* | (Mohm) | L* | A* | b* | (Mohm) |
| Control | 66.27 | −23.09 | 51.27 | 0.9 | 66.69 | −22.21 | 52.84 | 0.4 |
| (no Metal) | | | | | | | | |
| 0.5% Sn | 63.62 | −21.35 | 49.84 | 0.9 | 77.98 | −12.57 | 48.99 | >60 |
| 0.5% Sn + | 56.06 | −24.66 | 42.94 | 1 | 56.26 | −23.68 | 44.17 | 0.5 |
| 0.5% Cu | | | | | | | | |
| 1% Cu | 53.08 | −24.93 | 43.45 | 1.1 | 47.93 | −26.19 | 35.35 | 0.8 |
| 1% Sn | 54.38 | −21.37 | 52.15 | 0.9 | 76.41 | −10.13 | 36.78 | >60 |

TABLE 6
Color and resistance change from hydrogen peroxide sterilization cycle

| | Before | | | | After | | | |
|---|---|---|---|---|---|---|---|---|
| | | | | R | | | | R |
| Sample | L* | a* | b* | (Mohm) | L* | A* | b* | (Mohm) |
| Control | 66.92 | −23.54 | 52.2 | 0.9 | 59.71 | −29.01 | 37.05 | 6 |
| (no Metal) | | | | | | | | |
| 0.5% Sn | 65.71 | −21.81 | 50.93 | 1.2 | 58.87 | −27.39 | 35.91 | 8 |
| 0.5% Sn + | 54.68 | −24.04 | 41.65 | 1 | 44.36 | −29.7 | 18.71 | 32 |
| 0.5% Cu | | | | | | | | |
| 1% Cu | 53.66 | −24.59 | 43.25 | 1 | 39.93 | −29.33 | 17.85 | 35 |
| 1% Sn | 64.64 | −20.74 | 51.49 | 0.9 | 57.25 | −27.42 | 35.68 | 4 |

### Experiment 3

The procedure of Example 1 was used to prepare PANI/PU coating solutions containing 1% and 0.5% of silver, copper, and tin nanopowder solutions, respectively. In a separate vial, the solution containing 1% tin and the solution containing 1% silver were added in equal amounts and mixed well. In another vial, the solution containing 1% tin and the solution containing 1% of copper were combined in equal amounts and mixed well. Each of these solutions were coated on 3 mil PET and tested as described in Example 1. Sample strips prepared by coating each of these solutions were sterilized with steam (AMSCO Lab 110, 134° C. for 3.5 minutes) and hydrogen peroxide (Sterrad 100S). Tables 7 and 8 show the electric resistances and color measurements before and after sterilization. The data show that the Sn nanopowder-containing sample is still responsive to steam sterilization while silver and copper nanopowder-containing samples are response to hydrogen peroxide as shown in previous Examples. One difference from Experiment 2 is that unlike copper, silver nanopowder does not affect tin performance in steam sterilization.

TABLE 7
Colors and resistances before and after steam
sterilization at 134° C. for 3.5 minutes

| | Before | | | | After | | | |
|---|---|---|---|---|---|---|---|---|
| | | | | R | | | | R |
| Sample | L* | a* | b * | (Mohm) | L* | a* | b* | (Mohm) |
| Control | 68.86 | −23.54 | 54.47 | 0.38 | 69.89 | −23.93 | 55.51 | 0.15 |
| (No metal) | | | | | | | | |
| 0.5% Sn + | 59.81 | −19.55 | 50.1 | 0.33 | 78.68 | −8.84 | 34.35 | >60 |
| 0.5% Ag | | | | | | | | |
| 0.5% Sn + | 48.54 | −20.41 | 39.39 | 0.45 | 55.28 | −16.19 | 53.07 | 0.077 |
| 0.5% Cu | | | | | | | | |
| 0.5% Sn | 65.95 | −22.02 | 53.69 | 0.28 | 79.54 | −14.13 | 66.1 | >60 |

TABLE 8
Colors and resistances before and after hydrogen peroxide sterilization cycle.

| | Before | | | | After | | | |
|---|---|---|---|---|---|---|---|---|
| | | | | R | | | | R |
| Sample | L* | a* | b* | (Mohm) | L* | a* | b* | (Mohm) |
| Control | 68.45 | −23.72 | 54.76 | 0.38 | 64.52 | −26.87 | 45.3 | 0.9 |
| (No metal) | | | | | | | | |
| 0.5% Cu | 62.49 | −23.57 | 62.49 | 0.62 | 52.52 | −28.91 | 21.94 | 6.6 |
| 0.5% Ag | 64.9 | −23.38 | 53.9 | 0.3 | 59.32 | −28.64 | 39.61 | 1.8 |
| 0.5% Ag + | 60.79 | −19.57 | 50.49 | 0.3 | 54.56 | −24.62 | 36.63 | 0.95 |
| 0.5% Sn | | | | | | | | |
| 0.5% Cu + | 48.72 | −20.33 | 39.3 | 0.5 | 39.32 | −25.14 | 19.17 | 8.7 |
| 0.5% Sn | | | | | | | | |

### Example 4

A 100 nm-thick layer of aluminum was vapor deposited on a 3 mil-thick PET film in a pattern having two 5 mm×5 mm pads with a 1 mm gap with two extended legs was created using a mask. The pads were then stripe coated with the parent formulation shown in Table 2 mixed with 0.5% copper nanopowder, prepared as described above. The coating solution was applied with a #24 Meyer bar and then heated in an oven at 145° C. for 10 minutes. The electric resistance of six of the prepared coating samples was measured using a multimeter with the two pins directly contacting the two electric measuring feet before and after subjecting the samples to a hydrogen peroxide sterilization cycle. Table 9 shows the resistance changes and the associated color change for each sample. The data show that the resistance of the coatings changed significantly after exposure to hydrogen peroxide on metal electrodes.

TABLE 9
Resistances before and after hydrogen peroxide sterilization cycle.

| Sample | Resistance | Resistance | Color |
|---|---|---|---|
| number | (before) (MΩ) | (after) (MΩ) | change |
| 1 | 0.37 | >60 | From green to dark emerald |
| 2 | 0.5 | >60 | From green to dark emerald |
| 3 | 0.45 | >60 | From green to dark emerald |
| 4 | 0.57 | >60 | From green to dark emerald |
| 6 | 0.95 | >60 | From green to dark emerald |

### Example 5

Tamper-evident RFID tags (modified by two 5 mm×5 mm aluminum pads extending from the IC as shown in FIG. 2) were coated with parent PANI solutions mixed with 0.5% tin nanopowder and 0.5% copper nanopowder, respectively. The tags read as “open” using a ThingMagic Pro RFID reader before coating. After the tin or the copper nanopowder-containing PANI solution was coated and heated as described as Example 1, the tags were read as “short” due to the conducting polymer coating bridging the gap between the two metal pads on the RFID tag. The Sn/PANI coated RFID was then wrapped in a Bowie-Dick test pack (inside of a stack of index cards) and sterilized at 134° C. for 3.5 minutes with a full sterilization cycle. After the Bowie-Dick test pack was processed, the RFID was read through the test pack as “open” again, indicating the steam triggered chemical reaction occurred on the RFID surface. Control samples without polymer coating always read as “open” before and after steam sterilization under the same conditions. The copper/PANI coated RFID tag was placed on a perforated silicon mat and laid on the bottom of a plastic sterilization tray, and the tray was then closed. Before exposure to hydrogen peroxide, the RFID was read as “short” due to the PANI/Cu coating. After the completion of hydrogen peroxide sterilization, the tray was removed from the sterilizer and the RFID tag was read through the tray as “open” again, demonstrating the remote sensing capability of an RFID device to detect the hydrogen peroxide sterilization process without accessing the sensor. A control sample without a polymer coating showed consistent “open” response from the RFID reader.

All references and publications cited herein are expressly incorporated herein by reference in their entirety into this disclosure. Illustrative embodiments of this disclosure are discussed and reference has been made to possible variations within the scope of this disclosure. For example, features depicted in connection with one illustrative embodiment may be used in connection with other embodiments of the disclosure. These and other variations and modifications in the disclosure will be apparent to those skilled in the art without departing from the scope of the disclosure, and it should be understood that this disclosure is not limited to the illustrative embodiments set forth herein. Accordingly, the disclosure is to be limited only by the claims provided below and equivalents thereof.

1. A sensor device comprising:
a first electrode and a second electrode, each of the first and second electrodes being electrically coupled to an electrical bridge;
the electrical bridge comprising:
a conductive polymer having a first impedance state and a second impedance state that is different than the first impedance state;
metal or metal containing particles; and
a polymeric binder, wherein the metal or metal containing particles and conductive polymer are dispersed in the polymeric binder.

2. The sensor device of claim 1, wherein the electrical bridge is configured such that conductive polymer changes from the first impedance state to the second impedance in response to a change in one or more environmental conditions.

3. The sensor device of claim 2, wherein the electrical bridge is configured such that conductive polymer changes from the first impedance state to the second impedance in response to a change in response to exposure to a sterilant.

4. The sensor device of claim 3, wherein the sterilant comprises steam or hydrogen peroxide.

5. (canceled)

6. The sensor device of claim 1, wherein the metal or metal containing particles comprise aluminum, tin, bismuth, nickel, lead, indium, chromium, gallium, iron, vanadium, cadmium, titanium, zirconium, nobelium, tungsten, thallium, germanium, lanthanides, or alloys thereof.

7. The sensor device of claim 1, wherein the metal or metal containing particles comprise magnesium, copper, cobalt, manganese, zinc, iron, silver, platinum, osmium, iridium, palladium, lead, ruthenium, rhodium, gold, chromium, iron, vanadium, cadmium, titanium, zirconium, nobelium, tungsten, thallium or their oxidates and complexes.

8. The sensor device of claim 1, wherein the first and second electrodes are electrically coupled to the electrical bridge such that when the conductive polymer is in the first impedance state, a first resistance is measurable across the first and second electrode, and when the conductive polymer is in the second impedance state a second resistance is measurable across the first and second electrode, and wherein the first resistance is different than the second resistance.

9. The sensor device of claim 1, wherein the conductive polymer comprises a repeat unit of: aniline, acetylene, pyrrole, phenylene, phenylene vinylene, phenylene ethynylene, phenylene sulfide, fluorene, pyrene, azulene, napthalene, carbazole, indol, thiophene, ethylene dioxythiophene, or combinations thereof.

10. The sensor device of claim 1, wherein the conductive polymer comprises polyaniline.

11. A sterilization system, the system comprising:
a sterilizer having a chamber configured to receive medical devices for sterilization; and
a sensor device of claim 1 disposed in the chamber.

12. A method, the method comprising:
providing a sensor device of claim 1;
exposing the sensor device to a sterilant in a sterilization process.

13. The method of claim 12, wherein the sterilant comprises steam or hydrogen peroxide.

14. A sensor device comprising:
a sterilant-responsive switch comprising:
a first electrode and a second electrode, each having a first end electrically coupled to the circuit and a second end;
a conductive polymer having a first state and a second state;
a conductive particle; and
a polymeric binder;
wherein the conductive polymer is capable of being converted from being in the first state to being in the second state when in contact with a sterilant.
